(19) United States
(12) Patent Application Publication
(10) Pub. No.: US 2026/0208154 A1
(43) Pub. Date: Jul. 23, 2026

(54) METHOD FOR THE PRODUCTION OF SUPERABSORBENTS

(71) Applicant: BASF SE, Ludwigshafen Am Rhein (DE)

(72) Inventors: Monte Alan PETERSON, Freeport, TX (US); Vernon Lynn ADEY, Freeport, TX (US)

(73) Assignee: BASF SE, Ludwigshafen Am Rhein (DE)

(21) Appl. No.: 19/138,185

(22) PCT Filed: Dec. 6, 2023
(86) PCT No.: PCT/EP2023/084414

§ 371 Date: Jun. 12, 2025

(30) Foreign Application Priority Data
Dec. 14, 2022 (EP) 22213322.5

(51) Int. Cl. B01J 20/26; A61L 15/60; B01J 20/08; B01J 20/28; B01J 20/32

(52) U.S. Cl. CPC B01J 20/267; A61L 15/60; B01J 20/08; B01J 20/28004; B01J 20/28016; B01J 20/321; B01J 20/3217; B01J 20/3268; B01J 20/3289; B01J 20/3293

(57) ABSTRACT

The present invention relates to a process for continuously producing superabsorbents, wherein superabsorbent particles are coated by spray application of a surface postcrosslinker solution, the coated superabsorbent particles are thermally surface postcrosslinked in a contact drier 1, the thermally surface postcrosslinked superabsorbent particles are cooled in a contact drier 2, and a particulate solid is metered into the product stream between contact drier 1 and contact drier 2.

The present invention relates to a process for continuously producing superabsorbents, wherein superabsorbent particles are coated by spray application of a surface postcrosslinker solution, the coated superabsorbent particles are thermally surface postcrosslinked in a contact drier 1, the thermally surface postcrosslinked superabsorbent particles are cooled in a contact drier 2, and a particulate solid is metered into the product stream between contact drier 1 and contact drier 2.

Superabsorbents are used to produce diapers, tampons, sanitary napkins and other hygiene articles, but also as water-retaining agents in market gardening. Superabsorbents are also referred to as water-absorbing polymers.

The production of superabsorbents is described in the monograph “Modern Superabsorbent Polymer Technology”, F. L. Buchholz and A. T. Graham, Wiley-VCH, 1998, pages 71 to 103.

In order to improve performance properties, for example gel bed permeability (GBP) and absorption under a pressure of 49.2 g/cm² (AUL0.7 psi), superabsorbent particles are generally surface postcrosslinked. This increases the level of crosslinking of the particle surface, which can at least partly decouple the absorption under a pressure of 49.2 g/cm² (AUL0.7 psi) and the centrifuge retention capacity (CRC). This surface postcrosslinking can be performed in the aqueous gel phase. Preferably, however, superabsorbent particles (base polymer), having been dried, ground and sieved off, are surface coated with a surface postcrosslinker and thermally surface postcrosslinked. Crosslinkers suitable for that purpose are compounds which can form covalent bonds with at least two carboxylate groups of the superabsorbent particles.

It was an object of the present invention to provide an improved process for coating surface postcrosslinked superabsorbent particles with particulate solids.

The object was achieved by a process for continuously producing superabsorbents by coating superabsorbent particles by spray application of a surface postcrosslinker solution, thermally surface postcrosslinking the coated superabsorbent particles in a contact drier 1 and cooling the thermally surface postcrosslinked superabsorbent particles in a contact drier 2, wherein the surface postcrosslinked superabsorbent particles are additionally coated with a particulate solid, the particulate solid is metered into the product stream between contact drier 1 and contact drier 2, contact drier 2 has two horizontal shafts having mixing tools, and the speed of the mixing tools corresponds to a Froude number of 0.005 to 0.25.

Examples of contact driers suitable for the continuous process of the invention include paddle driers and disk driers. In a contact drier, the materials to be dried are moved along a heated surface by means of a dynamic tool and relayered. Contact driers can also be used for cooling.

The speed of the mixing tools corresponds to a Froude number of preferably 0.01 to 0.21, more preferably 0.02 to 0.18, most preferably 0.04 to 0.15.

For mixers with horizontally mounted mixing tools, the Froude number is defined as follows:

$$Fr = \frac{{ω}^{2} r}{g}$$

with
   r: radius of the mixing tool
   ω: angular frequency
   g: acceleration due to gravity

The particulate solids may be metered into the product stream in the form of a dispersion in a gas stream.

The present invention is based on the finding that undisrupted metered addition of particulate solids in the cooler (contact drier 2), especially of aluminum trihydroxide, is possible only with difficulty. Undisrupted mixing-in is possible when the particulate solids are added directly to the product stream falling into the cooler.

The temperature of the superabsorbent particles in the spray application of the surface postcrosslinker solution is preferably from 30 to 80° C., more preferably from 35 to 75° C. and most preferably from 40 to 70° C.

The surface postcrosslinker solution comprises preferably from 0.001% to 2% by weight, more preferably from 0.01% to 1% by weight, most preferably from 0.03% to 0.7% by weight, of a surface postcrosslinker, based in each case on the superabsorbent particles. The surface postcrosslinker solution preferably further comprises from 0.5% to 5% by weight, more preferably from 1.0% to 4% by weight, most preferably from 1.5% to 3% by weight, of water, based in each case on the superabsorbent particles.

The superabsorbent particles are heated in the contact drier 1 to a temperature of preferably 110 to 220° C., more preferably of 120 to 210° C., most preferably of 130 to 200° C. The residence time of the superabsorbent particles in the contact drier 1 is preferably from 10 to 60 minutes, more preferably from 15 to 50 minutes and most preferably from 20 to 40 minutes.

The contact drier 1 and the connection to the contact drier 2 may be trace-heated and/or thermally insulated.

The amount of particulate solid used is preferably from 0.001% to 2.0% by weight, more preferably from 0.01% to 1.0% by weight, most preferably from 0.1% to 0.5% by weight, based in each case on the superabsorbent particles. The average particle size of the particulate solid is preferably from 0.1 to 100 μm, more preferably from 0.5 to 50 μm, most preferably from 1 to 25 μm. The average particle size is the volume-average particle size and can be ascertained by light scattering. A suitable particulate solid is aluminum trihydroxide.

The temperature of the superabsorbent particles on coating with the particulate solid is preferably less than 180° C., more preferably less than 160° C., most preferably less than 140° C.

The superabsorbent particles are cooled in the contact drier 2 to a temperature of preferably 30 to 80° C., more preferably 35 to 70° C., most preferably 40 to 60° C. The residence time of the superabsorbent particles in the contact drier 2 is preferably from 10 to 60 minutes, more preferably from 15 to 50 minutes and most preferably from 20 to 40 minutes.

The mixing tools of the contact drier 2 have a diameter of preferably 0.2 to 2 m, more preferably 0.4 to 1.2 m, most preferably 0.6 to 1.2 m. The speed of the mixing tools is preferably less than 25, more preferably less than 20 and most preferably less than 10 revolutions per minute.

The production of the superabsorbents is described in detail hereinafter:

The superabsorbents are produced by polymerizing a monomer solution and are typically water-insoluble.

The ethylenically unsaturated monomers bearing acid groups are preferably water-soluble, i.e. their solubility in water at 23° C. is typically at least 1 g/100 g of water, preferably at least 5 g/100 g of water, more preferably at least 25 g/100 g of water and most preferably at least 35 g/100 g of water.

Suitable monomers are, for example, ethylenically unsaturated carboxylic acids, such as acrylic acid, methacrylic acid and itaconic acid. Particularly preferred monomers are acrylic acid and methacrylic acid. Very particular preference is given to acrylic acid.

The ethylenically unsaturated monomers bearing acid groups have typically been partly neutralized. The neutralization is conducted at the monomer stage. This is typically accomplished by mixing in the neutralizing agent as an aqueous solution or else preferably as a solid. The degree of neutralization is preferably from 40 to 85 mol %, more preferably from 50 to 80 mol % and most preferably from 60 to 75 mol %, for which the customary neutralizing agents can be used, preferably alkali metal hydroxides, alkali metal oxides, alkali metal carbonates or alkali metal hydrogencarbonates and also mixtures thereof. Instead of alkali metal salts, it is also possible to use ammonium salts. Particularly preferred alkali metals are sodium and potassium, but very particular preference is given to sodium hydroxide, sodium carbonate or sodium hydrogencarbonate and also mixtures thereof, especially sodium hydroxide.

The monomers typically comprise polymerization inhibitors, preferably hydroquinone monoethers, as storage stabilizers.

Suitable crosslinkers are compounds having at least two groups suitable for crosslinking. Such groups are, for example, ethylenically unsaturated groups which can be polymerized free-radically into the polymer chain, and functional groups which can form covalent bonds with the acid groups of the monomer. In addition, polyvalent metal salts which can form coordinate bonds with at least two acid groups of the monomer are also suitable as crosslinkers.

Suitable crosslinkers are, for example, ethylene glycol dimethacrylate, diethylene glycol diacrylate, polyethylene glycol diacrylate, allyl methacrylate, trimethylolpropane triacrylate, triallylamine, tetraallylammonium chloride, tetraallyloxyethane, as described in EP 0 530 438 A1, di-and triacrylates, as described in EP 0 547 847 A1, EP 0 559 476 A1, EP 0 632 068 A1, WO 93/21237 A1, WO 03/104299 A1, WO 03/104300 A1, WO 03/104301 A1 and DE 103 31 450 A1, mixed acrylates which, as well as acrylate groups, comprise further ethylenically unsaturated groups, as described in DE 103 31 456 A1 and DE 103 55 401 A1, or crosslinker mixtures, as described, for example, in DE 195 43 368 A1, DE 196 46 484 A1, WO 90/15830 A1 and WO 02/032962 A2.

The amount of crosslinker is preferably 0.05% to 1.5% by weight, more preferably 0.1% to 1% by weight, most preferably 0.15% to 0.6% by weight, calculated in each case on the basis of the total amount of monomer used. With rising crosslinker content, the centrifuge retention capacity (CRC) falls and the absorption under a pressure of 21.0 g/cm² (AUL0.3 psi) passes through a maximum.

Initiators used may be all compounds which generate free radicals under the polymerization conditions, for example thermal initiators, redox initiators or photoinitiators. Suitable redox initiators are sodium peroxodisulfate/ascorbic acid, hydrogen peroxide/ascorbic acid, sodium peroxodisulfate/sodium bisulfite and hydrogen peroxide/sodium bisulfite. Preference is given to using mixtures of thermal initiators and redox initiators, such as sodium peroxodisulfate/hydrogen peroxide/ascorbic acid. The reducing component used is preferably the disodium salt of 2-hydroxy-2-sulfonatoacetic acid or a mixture of the sodium salt of 2-hydroxy-2-sulfinatoacetic acid, the disodium salt of 2-hydroxy-2-sulfonatoacetic acid and sodium bisulfite. Such mixtures are obtainable as Bruggolite® FF6 and Bruggolite® FF7 (Brüggemann Chemicals; Heilbronn; Germany).

The water content of the monomer solution is preferably from 40% to 75% by weight, more preferably from 45% to 70% by weight and most preferably from 50% to 65% by weight. As the water content rises, the energy expenditure in the subsequent drying rises and, as the water content falls, the heat of polymerization can only be removed inadequately.

The temperature of the monomer solution is preferably from 10 to 90° C., particularly preferably from 20 to 70° C., very particularly preferably from 30 to 50° C.

For optimal action, the preferred polymerization inhibitors require dissolved oxygen. The monomer solution can therefore be freed of dissolved oxygen before the polymerization by inertization, i.e. flowing an inert gas through, preferably nitrogen or carbon dioxide. The oxygen content of the monomer solution is preferably lowered before the polymerization to less than 1 ppm by weight, more preferably to less than 0.5 ppm by weight, most preferably to less than 0.1 ppm by weight.

Suitable reactors for the polymerization are, for example, kneading reactors or belt reactors. In the kneader, the polymer gel formed in the polymerization of an aqueous monomer solution or suspension is comminuted continuously by, for example, contrarotatory stirrer shafts, as described in WO 2001/038402 A1. Polymerization on the belt is described, for example, in DE 38 25 366 A1 and U.S. Pat. No. 6,241,928. Polymerization in a belt reactor forms a polymer gel which has to be comminuted, for example in an extruder or kneader.

To improve the drying properties, the comminuted polymer gel obtained by means of a kneader can additionally be extruded.

The polymer gel is then typically dried with an air circulation belt drier until the residual moisture content is preferably 0.5% to 10% by weight, more preferably 1% to 7% by weight and most preferably 2% to 5% by weight, the residual moisture content being determined by EDANA recommended test method No. WSP 230.2-05 “Mass Loss Upon Heating”. In the case of too high a residual moisture content, the dried polymer gel has too low a glass transition temperature T_{g} and can be processed further only with difficulty. In the case of too low a residual moisture content, the dried polymer gel is too brittle and, in the subsequent comminution steps, undesirably large amounts of superabsorbent particles with an excessively low particle size are obtained (“fines”). The solids content of the polymer gel before the drying is preferably from 25% to 90% by weight, more preferably from 35% to 70% by weight, most preferably from 40% to 60% by weight. Subsequently, the dried polymer gel is crushed and optionally coarsely comminuted.

Thereafter, the dried polymer gel is typically ground and classified, and the apparatus used for grinding may typically be single- or multistage roll mills, preferably two- or three-stage roll mills, pin mills, hammer mills or vibratory mills.

The average particle size of the superabsorbent particles removed as the product fraction is preferably from 150 to 850 μm, more preferably from 250 to 600 μm, very particularly from 300 to 500 μm. The average particle size of the product fraction may be determined by means of EDANA recommended test method No. WSP 220.2 (05) “Particle Size Distribution”, where the proportions by mass of the screen fractions are plotted in cumulative form and the average particle size is determined graphically. The average particle size here is the value of the mesh size which arises for a cumulative 50% by weight.

The properties of the superabsorbent particles are further improved by thermal surface postcrosslinking. Suitable surface postcrosslinkers are compounds comprising groups that can form covalent bonds with at least two carboxylate groups of the superabsorbent particles. Suitable compounds are, for example, polyfunctional amines, polyfunctional amido amines, polyfunctional epoxides, as described in EP 0 083 022 A2, EP 0 543 303 A1 and EP 0 937 736 A2, di- or polyfunctional alcohols, as described in DE 33 14 019 A1, DE 35 23 617 A1 and EP 0 450 922 A2, or β-hydroxyalkylamides, as described in DE 102 04 938 A1 and U.S. Pat. No. 6,239,230.

In a preferred embodiment of the present invention, polyvalent cations are applied to the particle surface in addition to the surface postcrosslinkers.

The polyvalent cations usable in the process of the invention are, for example, divalent cations such as the cations of zinc, magnesium, calcium and strontium, trivalent cations such as the cations of aluminum, iron, chromium, rare earths and manganese, tetravalent cations such as the cations of titanium and zirconium. Possible counterions are chloride, bromide, hydroxide, sulfate, hydrogensulfate, carbonate, hydrogencarbonate, nitrate, phosphate, hydrogenphosphate, dihydrogenphosphate and carboxylate, such as acetate and lactate. Aluminum hydroxide, aluminum sulfate and aluminum lactate are preferred.

The amount of polyvalent cation used is, for example, 0.001% to 1.5% by weight, preferably 0.005% to 1% by weight and more preferably 0.02% to 0.8% by weight, based in each case on the polymer.

The surface postcrosslinking is performed in such a way that a solution of the surface postcrosslinker is sprayed onto the dried superabsorbent particles. After the spray application, the surface postcrosslinker-coated superabsorbent particles are subjected to thermal surface postcrosslinking.

The spray application of a solution of the surface postcrosslinker is preferably performed in mixers with moving mixing tools, such as screw mixers, disk mixers and paddle mixers. Particular preference is given to horizontal mixers such as paddle mixers, very particular preference to vertical mixers. The distinction between horizontal mixers and vertical mixers is made by the position of the mixing shaft, i.e. horizontal mixers have a horizontally mounted mixing shaft and vertical mixers have a vertically mounted mixing shaft. Suitable mixers are, for example, horizontal Pflugschar® plowshare mixers (Gebr. Lödige Maschinenbau GmbH; Paderborn; Germany), Vrieco-Nauta continuous mixers (Hosokawa Micron BV; Doetinchem; the Netherlands), Processall Mixmill mixers (Processall Incorporated; Cincinnati; USA) and Schugi Flexomix® (Hosokawa Micron BV; Doetinchem; the Netherlands). However, it is also possible to spray on the surface postcrosslinker solution in a fluidized bed.

The surface postcrosslinkers are typically used in the form of an aqueous solution. The penetration depth of the surface postcrosslinker into the superabsorbent particles can be adjusted via the content of nonaqueous solvent and total amount of solvent.

The thermal surface postcrosslinking is performed in contact driers, more preferably paddle driers, most preferably disk driers. Suitable driers are, for example, Hosokawa Bepex® Horizontal Paddle Dryer (Hosokawa Micron GmbH; Leingarten; Germany), Hosokawa Bepex® Disk Dryer (Hosokawa Micron GmbH; Leingarten; Germany), Holo-Flite® driers (Metso Minerals Industries Inc.; Danville; USA) and Nara Paddle Dryer (NARA Machinery Europe; Frechen; Germany).

Subsequently, the surface postcrosslinked superabsorbent particles can be classified again, with excessively small and/or excessively large superabsorbent particles being removed and recycled into the process.

The properties of the surface postcrosslinked superabsorbent particles can be further improved by coating or remoisturizing.

Remoisturizing is preferably performed at 30 to 80° C., more preferably at 35 to 70° C., most preferably at 40 to 60° C. At excessively low temperatures, the superabsorbent particles tend to form lumps, and, at higher temperatures, water already evaporates to a noticeable degree. The amount of water used for remoisturizing is preferably from 1% to 10% by weight, more preferably from 2% to 8% by weight and most preferably from 3% to 5% by weight. Remoisturizing increases the mechanical stability of the superabsorbent particles and reduces their tendency to static charging. Remoisturizing is advantageously performed in a cooler after the thermal surface postcrosslinking.

Suitable coatings for improving the swell rate and the gel bed permeability (GBP) are, for example, inorganic inert substances, such as water-insoluble metal salts, organic polymers, cationic polymers and di- or polyvalent metal cations. Suitable coatings for dust binding are, for example, polyols. Suitable coatings for counteracting the undesired caking tendency of the superabsorbent particles are, for example, fumed silica, such as Aerosil® 200, precipitated silica, such as Sipernat® D17, and surfactants, such as Span® 20.

### EXAMPLES

### Example 1 (Inventive)

By continuously mixing deionized water, 50% by weight sodium hydroxide solution and acrylic acid, a monomer solution was prepared such that the degree of neutralization corresponded to 71.0 mol %. The water content of the monomer solution was 60.5% by weight.

The crosslinker used was 3-tuply ethoxylated glyceryl triacrylate (purity about 85% by weight). The amount used was 1.42 kg per t of monomer solution.

The free-radical polymerization was initiated using, per t of monomer solution, 0.91 kg of a 0.25% by weight aqueous hydrogen peroxide solution, 4.30 kg of a 15% by weight aqueous sodium peroxodisulfate solution and 0.84 kg of a 1% by weight aqueous ascorbic acid solution.

The monomer solution was introduced into a List Contikneter continuous kneader reactor with a capacity of 6.3 m³ (LIST AG, Arisdorf, Switzerland). The throughput of the monomer solution was about 22 t/h. The reaction solution had a feed temperature of 23.5° C.

Between the addition point for the crosslinker and the addition sites for the hydrogen peroxide and sodium peroxodisulfate solutions, the monomer solution was inertized with nitrogen. Ascorbic acid was metered directly into the reactor.

After about 50% of the residence time, an additional about 1000 kg/h of superabsorbent particles obtained in the production process by comminution and classification, having a particle size of less than 150 μm, was metered into the reactor. The residence time of the reaction mixture in the reactor was about 15 minutes.

The polymer gel obtained was applied to the conveyor belt of an air circulation belt drier by means of an oscillating conveyor belt. The air circulation belt drier had a length of 48 m. The conveyor belt of the air circulation belt drier had an effective width of 4.4 m. On the air circulation belt drier, the aqueous polymer gel was exposed to a continuous flow of an air/gas mixture (about 175° C.) and dried. The dwell time in the air circulation belt drier was 37 minutes.

The dried polymer gel was comminuted by means of a three-stage roll mill and sieved off to a particle size of 150 to 850 μm. Superabsorbent particles having a particle size of less than 150 μm were separated off. Superabsorbent particles having a particle size of greater than 850 μm were recycled into the comminution. Superabsorbent particles having a particle size in the range from 150 to 850 μm were thermally surface postcrosslinked.

The superabsorbent particles were coated with a surface postcrosslinker solution in a Schugi Flexomix® (Hosokawa Micron B.V., Doetinchem, the Netherlands) and then thermally surface postcrosslinked in a NARA Paddle Dryer (contact drier 1, GMF Gouda, Waddinxveen, the Netherlands) at 120° C. for 45 minutes.

The following amounts were metered into the Schugi Flexomix®:

| | | |
|---|---|---|
| 9.5 | t/h | of superabsorbent particles |
| 530.10 | kg/h | of surface postcrosslinker solution |

The surface postcrosslinker solution comprised 1.43% by weight of ethylene glycol diglycidyl ether, 44.8% by weight of propane-1,2-diol and 53.77% by weight of water.

The surface postcrosslinked superabsorbent particles were transferred with a star feeder to a NARA Paddle Cooler (contact drier 2, GMF Gouda, Waddinxveen, the Netherlands) and cooled to about 60° C. This coated the surface postcrosslinked superabsorbent particles with a mixture of about 285 kg/h of water and 23.75 kg/h of a 1% by weight aqueous solution of sorbitan monolaurate (Span®20). The mixture was injected into the product bed from below into the mixer trough. The distance of the metering point from the end wall was about 200 cm. The paddles had a diameter of about 0.9 m. These rotated at about 10 revolutions per minute. The residence time was about 20 minutes.

The product stream falling from the star feeder into the NARA Paddle Cooler was at a temperature of about 120° C. The connecting tube between star feeder and NARA Paddle Cooler was extended into the NARA Paddle Cooler by means of an inserted tube. The inserted tube had a diameter of about 20 cm and a total length of about 50 cm, of which 30 cm projected into the NARA Paddle Cooler. The inserted tube should not project too far in order to avoid collision with mixing tools in the NARA Paddle Cooler. At the same time, the inserted tube within the NARA Paddle Cooler should not be too short either in order to prevent some of the aluminum trihydroxide from being drawn off with the output air via the gas space, and for the maximum amount of aluminum trihydroxide to remain in the product or on the product surface. A mixture of about 33.25 kg/h of aluminum trihydroxide (aluminum hydroxide dry gel, Dr. Paul Lohmann GmbH KG, Emmerthal, Germany) and 45 kg/h of air was metered into the product stream falling through the inserted tube about 20 cm from the lower end of the inserted tube. The aluminum hydroxide had an average particle size of about 20 μm.

The coating with aluminum trihydroxide was run without disruption.

### Example 2 (not According to the Invention)

The procedure was as in example 1. The mixture of aluminum trihydroxide and air was injected into the product bed from below into the mixer trough. The distance of the metering point from the end wall was about 180 cm.

Blockages occurred in the feed for the aluminum trihydroxide into the NARA Paddle Cooler.

1. A process for continuously producing superabsorbents, comprising:
coating superabsorbent particles by spray application of a surface postcrosslinker solution;
thermally surface postcrosslinking the coated superabsorbent particles in a first contact drier; and
cooling the thermally surface postcrosslinked superabsorbent particles in a second contact drier;
wherein:
the surface postcrosslinked superabsorbent particles are additionally coated with a particulate solid;
the particulate solid is metered into the product stream between the first contact drier and the second contact drier;
the second contact drier has two horizontal shafts having mixing tools; and
a speed of the mixing tools corresponds to a Froude number of 0.005 to 0.25.

2. The process according to claim 1, wherein the speed of the mixing tools corresponds to a Froude number of 0.04 to 0.15.

3. The process according to claim 1, wherein a residence time of the superabsorbent particles in the second contact drier is 10 to 60 minutes.

4. The process according to claim 1, wherein the particulate solid has an average particle size of 1 to 25 μm.

5. The process according to claim 1, wherein the particulate solid is used in an amount of 0.001% to 2.0% by weight, based on a total weight of the superabsorbent particles.

6. The process according to claim 1, wherein the mixing tools have a diameter of 0.2 to 2 m.

7. The process according to claim 1, wherein the speed of the mixing tools is less than 25 revolutions per minute.

8. The process according to claim 1, wherein a temperature of the superabsorbent particles on coating with the particulate solid is less than 180° C.

9. The process according to claim 1, wherein the superabsorbent particles are cooled in the second contact drier to a temperature of 30 to 80° C.

10. The process according to claim 1, wherein the particulate solid comprises aluminum trihydroxide.

11. The process according to claim 1, wherein the superabsorbent particles comprise partly neutralized, crosslinked polyacrylic acid.

12. The process according to claim 1, wherein the surface postcrosslinker is capable of forming covalent bonds with the superabsorbent.

13. The process according to claim 1, wherein a temperature of the superabsorbent particles in the spray application of the surface postcrosslinker solution is 30 to 80° C.

14. The process according to claim 1, wherein the superabsorbent particles are heated in the first contact drier to a temperature of 110 to 220° C.

15. The process according to claim 1, wherein a residence time of the superabsorbent particles in the first contact drier is 10 to 60 minutes.
